(19) United States
(12) Patent Application Publication
(10) Pub. No.: US 2023/0404768 A1
(43) Pub. Date: Dec. 21, 2023

(54) TOTAL ANKLE REPLACEMENT WITH ANATOMICALLY FITTED TALAR COMPONENT

(71) Applicant: DREXEL UNIVERSITY, Philadelphia, PA (US)

(72) Inventor: Sorin Siegler, Narberth, PA (US)

(73) Assignee: DREXEL UNIVERSITY, Philadelphia, PA (US)

(21) Appl. No.: 18/450,091

(22) Filed: Aug. 15, 2023

Related U.S. Application Data
(63) Continuation of application No. 16/563,197, filed on Sep. 6, 2019.
(60) Provisional application No. 62/731,217, filed on Sep. 14, 2018.

(51) Int. Cl. A61F 2/42; A61F 2/30

(52) U.S. Cl. CPC A61F 2/4202; A61F 2/30942; A61F 2002/4207; A61F 2002/4205; A61F 2002/30943; A61F 2310/00017; A61F 2310/00179; A61F 2310/00023; A61F 2002/30985; C08L 2207/068

(57) ABSTRACT

An anatomically fitted talar component for use in an ankle replacement system having a body including a talar surface having a portion contoured to approximately or exactly fit with a surface portion of a three dimensional rendering of bone of a talar dome; and a tibial surface configured for forming a joint with a second component of the ankle replacement system. A method of forming the talar component by: (i) obtaining image data of the talar dome, (ii) using the data to create a three-dimensional model of the talar dome, and (iii) forming a body having a talar surface that approximately or exactly fits with a portion of the surface of the three-dimensional model.

### CROSS-REFERENCE TO RELATED APPLICATIONS

This application is a continuation of U.S. patent application Ser. No. 16/563,197, filed on Sep. 6, 2019, which, in turn, claims the benefit of U.S. provisional application No. 62/731,217, filed on Sep. 14, 2018, the entire disclosures of which are hereby incorporated by reference herein.

### FIELD OF THE INVENTION

The invention relates to a total ankle replacements and methods for making them. More specifically, the invention relates to a talar component of a total ankle replacement that is configured to approximately or exactly fit with the bone of the talus of the patient and to methods for making and attaching the talar component to the bone of the patient's talus.

### BACKGROUND OF THE INVENTION

For many years there has been considerable interest and activity with respect to ankle joint replacement, in which the degenerative articular surfaces are removed and replaced with an artificial joint called an ankle joint prosthesis. This is used as a treatment of diseased or injured ankle joints. As the population ages, the demand for ankle joint prostheses is growing.

Fusion has long been an alternative to ankle arthroplasty but fusion has drawbacks. For example, there is a loss of motion in the ankle joint which may cause difficulties with associated parts of the foot and leg. More recent research on the ankle joint has allowed for improved designs for ankle joint prostheses and better implant materials allowing ankle joint prostheses to dramatically improve in quality and longevity. Many types of ankle joint prostheses have been developed over the past thirty years.

Fixation of the talar component of a total ankle replacement (TAR) to the talus is a major problem faced by present day TARs. Resection of this small bone and fixation to weak cancellous bone produces a weak bone-implant interface that often results in collapse or shift of the talar component of the TAR from its original position.

U.S. Patent Application Publication No. 2017/0181861 discloses an ankle replacement with a talar implant. The talar-facing surface of the talar implant has three generally planar surfaces that match to the planar surfaces created on the top of the talus.

U.S. Patent Application Publication No. 2017/0340450 discloses an ankle replacement for use in treating degenerative conditions of the ankle. The talar component specifically includes a large undersurface having a wide planar area for bony ingrowth. This wide planar surface is said to reduce loosening of the talar component due to its large size and the increased osseo-integration provided by the wide area for bony ingrowth.

U.S. Pat. No. 9,750,613 discloses an ankle prosthetic having a tibial component and a talar component. The talar component has a flat surface used for attachment to the talus. The flat surface is provided with attachment means used to secure the surface to the bones. The attachment means may include screws or other devices.

U.S. Pat. No. 6,409,767 discloses an ankle joint prosthesis comprising a talar implant for implanting in or on the talus and a top element including a tibial implant for implanting in or on the base of the tibia. The top element and the talar implant are mounted to move relative to each other, which movement is impeded by friction on a contact interface so as to allow the ankle to move. The contact interface presents a friction surface that can be considered a portion of a substantially frustoconical surface. When implanted, the substantially frustoconical surface is oriented so that its larger radius portion is directed substantially towards the lateral side of the ankle in accordance with the postulate of Inman's Joints. The top surface of the talar implant has two ribs on both edges running from the anterior to the posterior edges.

One example of an ankle joint prosthesis is disclosed in U.S. Pat. No. 7,025,790, which describes an ankle joint prosthesis comprising tibial, talar and mobile or semi-constrained bearing components that may be implanted in a patient. The top surface of the tibial component has a convex curvature and is configured so as to approximate and fit with the curvature of a prepared portion of the distal tibia. The bottom surface of the tibial component is approximately flat. The top surface of the talar component has a saddle-shaped, convex curvature in its anterior to posterior plane. The bottom surface of the talar component has a concave curvature and is configured so as to approximate and fit with the curvature of a prepared portion of the talus.

WO 2006/023824 discloses an ankle joint prosthesis including a talar component having a lower surface with a bone fixation portion for fixation to the talus and an upper surface designed for articulation using a bearing component. The bearing component can have a lower surface for articulation relative to the talar component and an upper surface for articulation relative to the tibial component.

After initial encouraging results, follow-up clinical studies on many of these ankle joint prostheses revealed frequent failures of such implants due mainly to the inadequate restoration of the natural mobility and the poor stability of the resulting ankle implants. Many of the problems originated from instability produced by the connection between the implant and the cancellous bone of the talus. In each of the above disclosures the implant used to replace the surface of the talus requires preparation of the talus surface, including removal of a significant portion, if not all, of the subchondral bone of the talus. The resulting interface lacks rigidity, and frequently becomes unstable over time.

One objective of the present invention is to provide an improved talar component for use with a TAR. The improved talar component of the present invention is designed to be attached in a certain way to the anatomical structure of the existing talus to provide greater stability to the joint implant over time.

### SUMMARY OF THE INVENTION

An anatomically fitted talar component for use in an ankle replacement system. The talar component includes a body having a talar surface having a portion contoured to approximately or exactly fit with a surface portion of a three dimensional rendering of bone of a talar dome; and a tibial surface configured for forming a joint with a second component of the ankle replacement system

In the foregoing embodiment of the talar component, the three dimensional rendering may include cortical bone of the talar dome.

In each of the foregoing embodiments, the three dimensional rendering may include a portion of cancellous bone of the talar dome that is exposed by resection of a portion of cortical bone of the talar dome.

In each of the foregoing embodiments, the portion of the talar surface may approximately or exactly fit with a resected surface portion of the corresponding surface portion of the bone of the talar dome. In this embodiment, the three dimensional rendering may be altered to compensate for injury to or disease of the talus, and, optionally, the bone of the talar dome may be altered to compensate for injury to or disease of the talus.

In each of the foregoing embodiments, the talar surface may further include at least one protrusion shaped to fit a resected portion of bone of the talar dome.

In each of the foregoing embodiments, the portion of the talar surface may exactly fit with the corresponding surface portion of the three dimensional rendering of the bone of the talar dome.

In each of the foregoing embodiments, the portion of the talar surface may approximately fit with the corresponding surface portion of the three dimensional rendering of the bone of the talar dome or approximately fit with the corresponding surface portion of the bone of the talar dome.

In another embodiment, the invention relates to a method of attaching the talar component of each of the foregoing embodiments to a talus. The method may include steps of:
shaving one or more of articular cartilage, osteophytes, and non-conforming portions of the talar dome to expose bone of the talar dome, wherein the non-conforming portions of the talar dome represent less than 75% of the surface area of the talar component;
creating one or more recesses in the subchondral bone of the talar dome to correspond to one or more protrusions located on the talar surface of the talar component; and
securing the talar surface of the talar component to the talar dome with the protrusions located in the recesses.

In one embodiment of the foregoing method no resection of the talus may be carried out other than creating the recesses.

In another embodiment of the foregoing method, a portion of the bone of the talar dome may be resected to compensate for injury to or disease of the talus prior to securing the talar surface of the talar component to the talar dome.

In a third embodiment, the present invention relates to a method of forming a talar component of an ankle replacement system. The method may include steps of:
obtaining image data of talar dome;
using the obtained image data to create a three-dimensional model of the talar dome; and
forming a body having a talar surface that approximately or exactly fits with a portion of the surface of the three-dimensional model.

The foregoing third embodiment may further include a step of modifying a portion of the talar surface of the three dimensional model of the talar dome to compensate for injury to or disease of the talus prior to forming the body.

The foregoing third embodiment may further include a step of altering the image data to compensate for injury or disease on the surface of the talar dome prior to using the image data to create the three dimensional model of the talar dome.

The foregoing third embodiment may further include a step of creating at least one recess in a surface of the three dimensional model prior to forming the body. This embodiment of the method may further include a step of altering the image data to include the at least one recess prior to using the image data to create the three dimensional model of the talar dome.

Each of the foregoing third embodiments may further include a step of forming at least one protrusion on a portion of the talar surface of the body. In this embodiment, each said at least one protrusion may align with at least said recess in the surface of the three dimensional model.

### BRIEF DESCRIPTION OF THE FIGURES

FIG. 1 depicts a human talus and identifies the lateral and medial sides as well as the anterior and posterior directions as used in the description of the present application.

FIG. 2 is a sagittal plane cross-section of a talus on the medial side taken along medial plane 1 of FIG. 1, shown with a medial circle that represents a best fit to the radius of curvature of the top surface of the talus shown in the cross-section taken in medial plane 1.

FIG. 3 is a sagittal plane cross-section of a talus on the lateral side taken along lateral plane 2 of FIG. 1, shown with a lateral circle that represents a best fit to the radius of curvature of the top surface of the talus shown in the cross-section taken in lateral plane 2.

FIG. 4 is a sagittal plane cross-section of a talus at a central location taken along central plane 3 of FIG. 1, shown with a central circle that represents a best fit to the radius of curvature of the top surface of the talus shown in the cross-section taken in the central plane 3.

FIG. 5A depicts the medial circle, lateral circle and central circle of FIGS. 2-4 that are used to model the top surface of the human talus showing the radius of each of the circles.

FIG. 5B depicts a conical surface used to model the talus and which is formed using the medial, lateral and central circles shown in FIG. 5A.

FIG. 6 shows the conical surface of FIG. 5B superimposed on a human talus.

FIG. 7 shows a cone that may be used to model a human talus.

FIG. 8A depicts a tibial component of a prosthetic ankle according to one embodiment of the present invention.

FIG. 8B depicts a talar component of a prosthetic ankle according to one embodiment of the present invention.

FIG. 9 shows a frontal plane cross-sectional view of a talar component in accordance with one embodiment of the invention fit on top of a human talus.

FIG. 10 shows multiple frontal plane cross-sectional views of a talar component in accordance with one embodiment of the invention.

FIG. 11 shows a top perspective view of one embodiment of a talar component fitted onto the frustoconical surface of FIGS. 5A-5B and 6 which models a top surface of the human talus.

FIG. 12 shows a bottom perspective view of one embodiment of a talar component of the invention with spikes on the posterior end and a ridge on the anterior end.

FIG. 13 shows a top perspective view of another embodiment of talar component of the invention with holes on the posterior end which may be used to affix the talar component to the talus using screws or other affixation devices.

FIG. 14 shows a prosthetic ankle according to another embodiment of the present invention.

FIG. 15A shows an alternative embodiment of the tibial component of the present invention,

FIG. 15B shows an embodiment of a bearing component of a prosthetic ankle according to one embodiment of the present invention adapted for use with the tibial component of FIG. 15A.

FIG. 16A shows a tibial component with a flat bottom surface according to one embodiment of the present invention.

FIG. 16B shows a bearing component with a flat top surface adapted for use with the tibial component of FIG. 16A according to one embodiment of the present invention.

FIG. 17 shows an alternative model for defining a conical surface used to describe the top surface of the talus.

FIG. 18 depicts a cone used as a model for some prior art designs of ankle implants.

FIG. 19 depicts a cross-sectional view taken on the lateral side of the talus showing a circle centered about the assumed axis of rotation of the prior art comparative model of FIG. 18.

FIG. 20 depicts a cross-sectional view taken on the medial side of the talus showing a circle centered about the assumed axis of rotation of the prior art comparative model of FIG. 18.

FIG. 21 depicts a cone generated by a surface that is tangent to each of the circles of FIGS. 19-20.

FIG. 22 is a computer generated image of a total ankle replacement system comprising an anatomically fitted talar component.

FIG. 23 is a computer-generated image of a human ankle having the system of FIG. 22.

FIG. 24 is an side view of the computer-generated image of FIG. 23 without the fibula of the ankle shown.

FIG. 25 shows the anatomically fitted talar component depicted in FIG. 22 and a three-dimensional rendering of a talar head.

FIG. 26 shows a perspective view of the anatomically fitted talar component shown in FIG. 25

FIG. 27A shows the tibial-side view of the anatomically fitted talar component of FIG. 26.

FIG. 27B shows the talar-side view of the anatomically fitted talar component of FIG. 26.

FIG. 28 shows three-dimensional rendering of the talar surface of the talar component shown in FIG. 25.

FIG. 29 is a perspective view of the talar component of FIG. 25 located on the talar head of FIG. 25.

FIG. 30A is underside view of a computer-generated image of an alternative embodiment of the anatomically fitted talar component.

FIG. 30B shows a top view of the anatomically fitted talar component shown in FIG. 30A.

FIG. 30C is a three-dimensional rendering of a talar head as prepared for use with the anatomically fitted talar component shown in FIG. 30A.

FIG. 30D is a computer-generated image of the talar component of FIG. 30A located on the talar head shown in FIG. 30C.

FIG. 31 is depicts a template for resecting a portion of the talus.

FIG. 32 shows resected recesses in a talus.

FIG. 33 shows a talar component secured on the talus.

FIG. 34 depicts a total ankle replacement system comprising an anatomically-fitted talar component.

### DETAILED DESCRIPTION OF EMBODIMENTS OF THE INVENTION

This disclosure proposes a talar component for a TAR that attaches to the superior part of the talus leaving as much of the talus intact as possible. The primary objective is to secure the talar component to the talus with no resection or as little resection of the talus as possible. The process of creating the talar component may begin with a computer tomography (CT) scan or magnetic resonance imaging (MRI) of the ankle from which a three dimensional rendering of the talus is obtained.

The talar component of the TAR is then fabricated with a surface that approximately or exactly fits the three-dimensional rendering of the shape of a surface portion of the bone of the talar dome of at least a section of a talus body that interfaces with the talar component. Specifically, the talar component is designed with a surface that approximately or exactly fits with the top surface of the talus that articulates with the distal tibia.

Optionally, one or more protrusions, ridges or pegs for anchoring the talar component to the subchondral bone may protrude from the surface of the talar component. These protrusions, ridges or pegs are used to fix the talar component to the subchondral bone of the talar dome and to provide surfaces for bone growth that will improve long-term fixation of the talar component to the sub chondral bone of the talus.

In a preferred embodiment using this TAR, implantation of the talar component involves shaving or removal of the articular cartilage on the talar dome to expose the bone and optional preparation of recesses in the talar dome for receiving the protrusions or pegs of the talar component. No resection of the talar bone is required for placement of this talar component. The invention can be applied to any TAR with any surface geometry.

This invention does not require resection of the superior part of the talus, which is part of the small talar bone. Resection of this small bone greatly weakens the bone and thus the present invention desires to avoid or minimize resection of this bone for fixation of the talar component since weakening of the talar bone by resection may result in migration and/or failure of the TAR over time, after implantation. The invention mitigates this problem by not requiring resection the talar bone for implantation of the TAR. This is typically accomplished by customizing the interfacial surface of the talar component of the implant to fit with the surface of the bone of a patient's talus, without resection of the talar bone. Since the talar bone is not resected during this process, the TAR does not disrupt the structural integrity of the talar bone leading to a stronger bone-implant fixation and a reduction in short and long term failure rates of the TAR.

In one embodiment, the three dimensional geometry of the talar dome from image data from a particular patient may be used to create a three dimensional rendering of the actual patient's talus. This may be particularly desirable if it is foreseen that some corrective modification of the talar dome of the patient will be carried out prior to the ankle replacement since it will permit customization of both the three dimensional rendering and the talar component to take into account proposed modifications to the talar dome.

For illustrative purposes, the principles of the present invention are described by referencing various exemplary embodiments. Although certain embodiments of the invention are specifically described herein, one of ordinary skill in the art will readily recognize that the same principles are equally applicable to, and can be employed in other systems and methods. Before explaining the disclosed embodiments of the present invention in detail, it is to be understood that the invention is not limited in its application to the details of any particular embodiment shown. Additionally, the terminology used herein is for the purpose of description and not of limitation. Furthermore, although certain methods are described with reference to steps that are presented herein in a certain order, in many instances, these steps may be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Furthermore, the terms “a” (or “an”), “one or more” and “at least one” can be used interchangeably herein. The terms “comprising”, “including”, “having” and “constructed from” can also be used interchangeably.

All references to frontal plane cross-sections are to be interpreted as references to coronal plane cross-sections as these terms are used interchangeably in the present application.

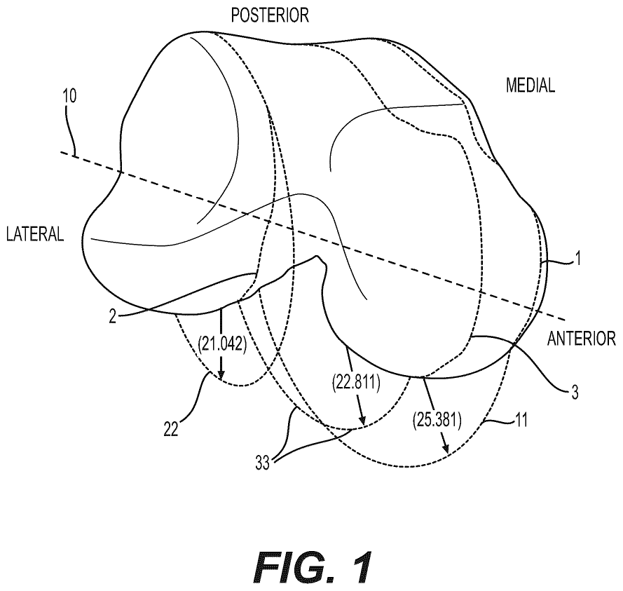

Referring to FIG. 1, there is shown a human talus. The anterior is the front end of the talus in the direction of the toes. The posterior is the back end of the talus in the direction of the heel. The lateral side refers to the outside of the talus of a foot or an ankle, i.e. the side that faces away from the other foot or ankle. The medial side refers to the inside of the talus of a foot or an ankle, i.e. the side that faces toward the other foot or ankle. All references to orientation in this application are given based on the orientation of the prosthetic ankle when implanted in a human. References to the top or to above the device refer to a direction towards the head of a human whereas references to the bottom or below the device refer to a direction towards the bottom of the foot of a human.

Implantation of the device of the invention is typically done from the anterior side of the ankle. Prior to implantation of the prosthetic ankle, the lower surface of the tibia and the upper surface of the talus may be prepared to receive the device by, for example, shaping these surfaces to a desired, predetermined shape. For example, the curvature of each of the lower surface of tibia and the upper surface of talus may be adapted to receive a particular prosthetic ankle by, for example, shaping these surfaces to approximate the shape of adjacent surfaces of the tibial and talar components 100, 300 of the prosthetic ankle, respectively. Thus, the tibial component 100 may be adapted to fit snugly onto the shaped lower surface of the tibia and the talar component 300 may be adapted to fit snugly onto the shaped upper surface of the talus.

The tibial component 100 of the present invention is designed to be joined to the tibia during the implantation procedure using conventional joining means such as adhesives, screws, spikes, friction fit, form fit and/or any combination thereof. The talar component 300 of the present invention is designed to be jointed to the talus during the implantation procedure using conventional joining means such as adhesives, screws, friction fit, form fit, spikes, and/or any combination thereof.

In one aspect, the present invention relates to a prosthetic ankle including a tibial component 100 and a talar component 300. The talar component 300 according to the present invention is specially designed using the natural curvature and shape of the top surface of a human talus as a basis for the design elements of talar component 300.

The design of a talar component 300 of the present invention is described in relation to FIGS. 1-6. Initially, three sagittal plane cross-sections are employed to model key aspects of the upper surface of the talus. Referring to FIG. 1, the three sagittal plane cross-sections are shown as the medial plane 1, the lateral plane 2, and the central plane 3.

The medial plane 1 is a sagittal plane cross-section taken on the medial side of the talus that passes through the peak of the medial talar trochlear shoulder. The medial plane 1 should follow the peak of the shoulder from anterior to posterior. The peak of the medial talar trochlear shoulder is defined as the point of inflection.

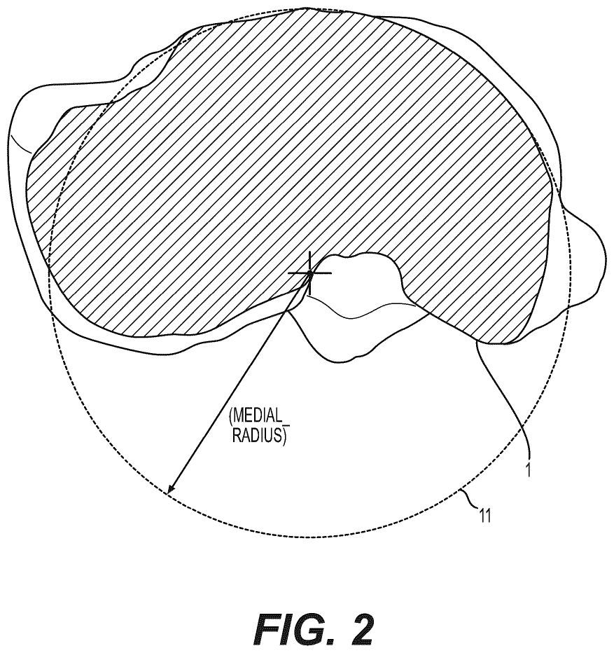

The cross-section of the talus bone at the medial plane 1 is shown in FIG. 2. The top portion of the cross-section shows the top surface curvature of the talus from the posterior end to the anterior end. In the next step of the process, a circle is best fit to at least a major portion of the top surface curvature of the talus in medial plane 1 to define a medial circle 11. The average measured radius of this medial circle 11 for a sampling of adult human tali was about 25.38 mm.

The sagittal plane cross-section used for the lateral plane 2 is located by first creating a plane that is parallel to the medial plane 1 with an offset from the medial plane 1 in the lateral direction. The offset should approximate the distance between the lateral trochlear shoulder and the medial plane 1. A suitable average lateral plane offset starting from medial plane 1 may be about 25 mm but can range from 20-30 mm, depending on the patient. The lateral plane 2 is then rotated about a projected superior-inferior line such that the resultant lateral plane 2 follows the peak of the lateral trochlear shoulder from an anterior location to a posterior location. A typical rotation of lateral plane 2 is from about 7-12 degrees with the average rotation being about 9.9 degrees.

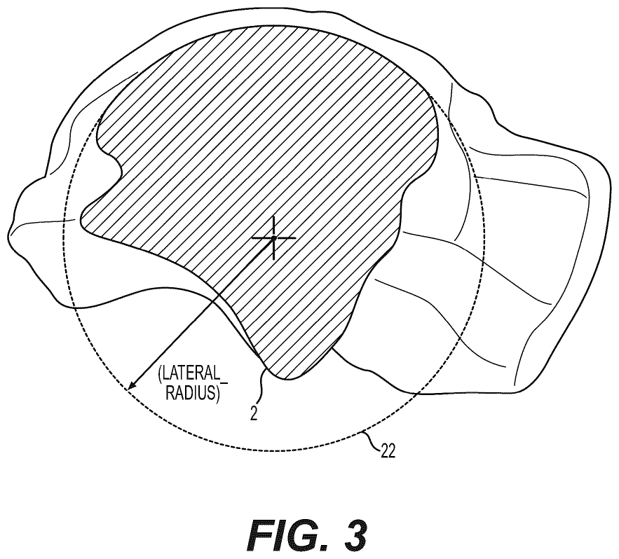

The cross-section of the talus bone at the lateral plane 2 is shown in FIG. 3. The top portion of the cross-section at the lateral plane 2 shows the top surface curvature of the talus from the posterior end to the anterior end as viewed in this sagittal plane cross-section. A circle is best fit to at least a major portion of the top surface of the lateral plane 2 to define a lateral circle 22. The average measured radius of the lateral circle 22 of a sampling of several adult human tali was about 21.04 mm.

The central plane 3 is located by first creating a plane that is parallel to the medial plane 1 with an offset from medial plane 1 in a lateral direction. The offset should approximate the change in curvature along the central portion of the trochlear surface from anterior to posterior. A suitable average central plane offset starting from medial plane 1 is about 10.5 mm and may vary from about 9-12 mm. Then this plane is rotated about the projected superior-inferior line such that the resultant central plane 3 follows the trough or valley of the medial to lateral concavity in the anterior to posterior direction. A typical rotation of central plane 3 is from about 1-7 degrees with the average rotation being about 4 degrees.

The cross-section of the talus bone at the central plane 3 is shown in FIG. 4. The top portion of the cross-section shows the curvature of the top surface of the talus from the posterior end to the anterior end as viewed in this sagittal plane cross-section. A circle is best fit to at least a major portion of the top surface curvature at the central plane 3 cross-section to define a central circle 33. The average radius of the central circle 33 was about 22.81 for a sampling of several adult human tali.

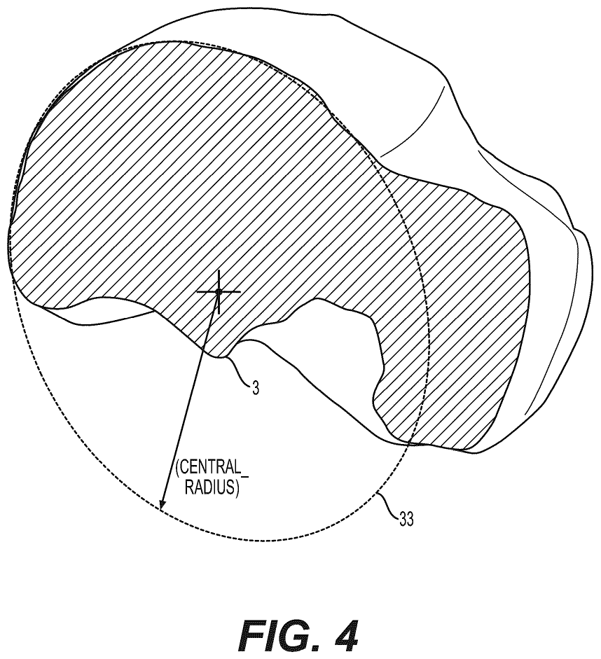

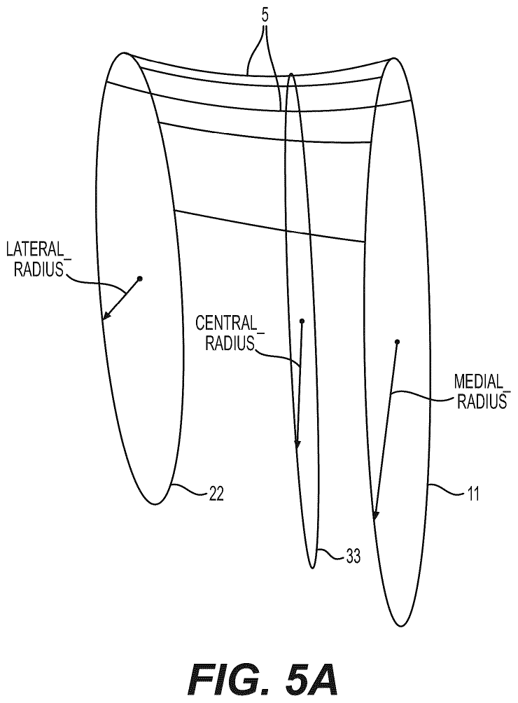

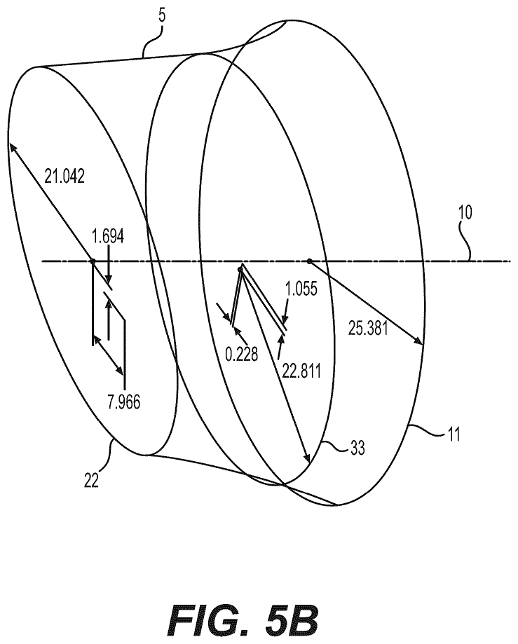

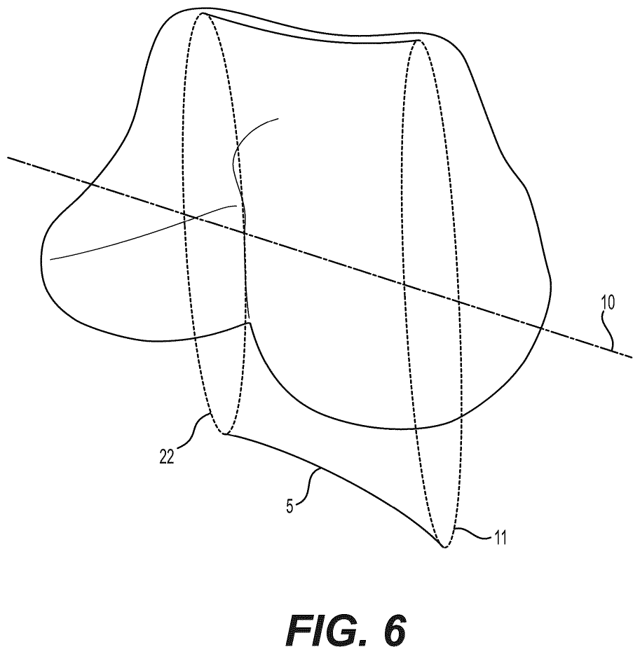

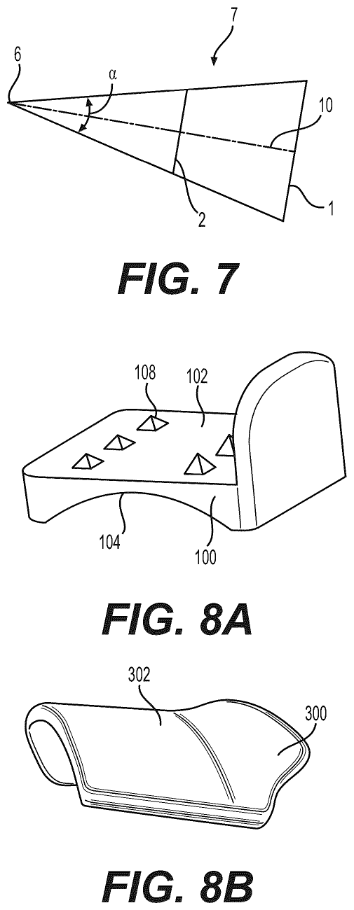

Referring to FIGS. 5A and 5B, the medial circle 11, lateral circle 22 and central circle 33 are used to model the top surface of a human talus. Thus, in FIG. 5A, the top portions of the three circles are connected to form a frustoconical surface that models the top surface of the human talus. Connections in five frontal plane cross-sections are shown in FIG. 5A. In FIG. 5B, the bottom surfaces of the medial circle 11, lateral circle 22 and central circle 33 are connected to form a truncated cone 5 that approximates the human talus. FIG. 6 shows the truncated cone 5 of FIG. 5B superimposed on a human talus. The truncated cone 5 of FIGS. 5B and 6 can be extended to generate a full cone 7 as shown in FIG. 7. The apex 6 of the cone 7 points in a substantially lateral direction due to the fact that the radius of the medial circle 11 is larger than the radius of the lateral circle 22, as described in greater detail below.

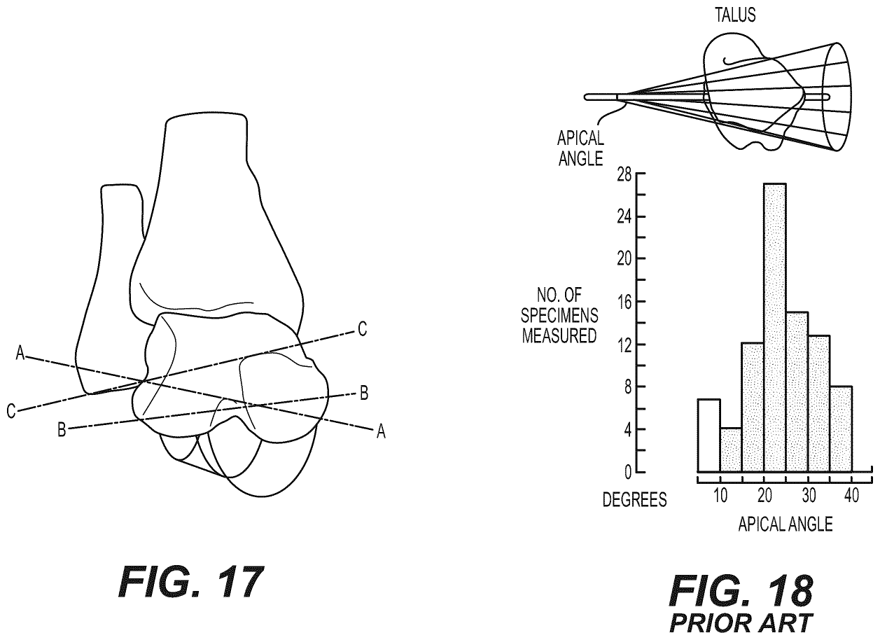

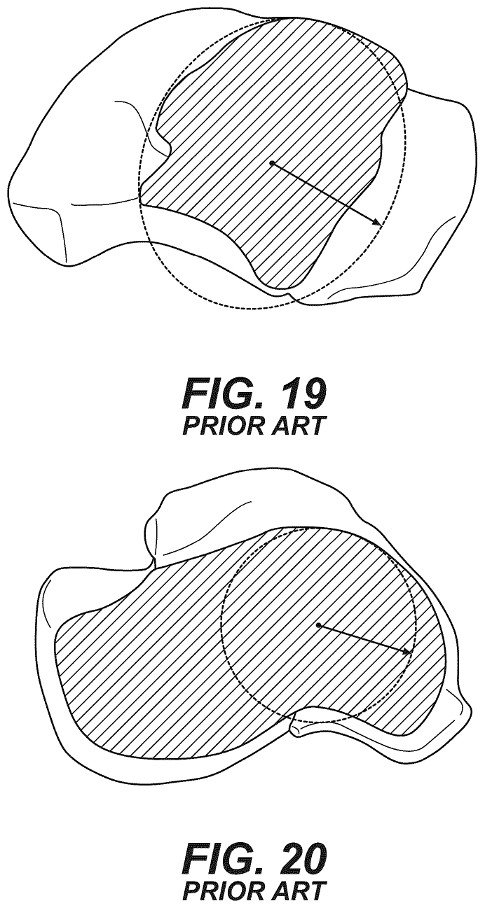

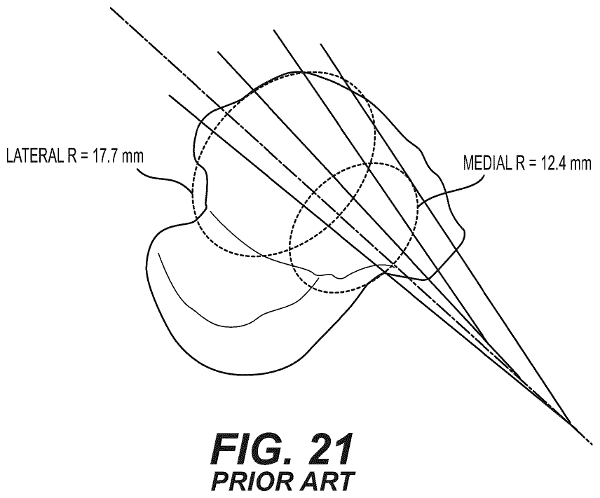

As shown in FIG. 6, a line drawn through the center of the medial circle 11 and the center of lateral circle 22 defines the medial-lateral axis 10 of both the truncated cone 5 and the full cone 7. Referring to FIG. 7, the cone 7 intersects with the medial plane 1 and lateral plane 2. The apex angle alpha of this cone 7 may be in the range of from 2° to 30°, or from 3° to 20°, or from 5° to Referring to FIG. 17, the talus may also be described using an alternative model. Line A-A of FIG. 17 connects the centers of the medial circle 11 and lateral circle 12, considered as the axis 10 of the cone 7 that resembles the top surface of the talus. Line B-B is a line perpendicular to the medial circle 11 and through its center. Line C-C is a line connecting the tips of the medial and lateral malleoli. In this alternative model, the angle between lines A-A and B-B, which is called total axis offset angle, is in a range from 0° to 40°, or from 5° to 35°, or from 10° to 30°, or from 16° to 24°. This angle when projected onto a coronal plane, which is called coronal axis offset angle, is in a range of from 0° to 38°, or from 6° to 32°, or from 10° to 28°, or from 15° to 23°. The angle between lines A-A and B-B when projected onto a transverse plane, which is called transverse axis offset angle, is in a range of from 0° to 20°, or from 4° to 16°, or from 6° to 14°. Further, the angle between line A-A connecting the centers of the medial circle 11 and lateral circle 12 and line C-C connecting the tips of the medial and lateral malleoli, is in a range from 5° to 25°, or from 10° to 20°, or from 13° to 20°.

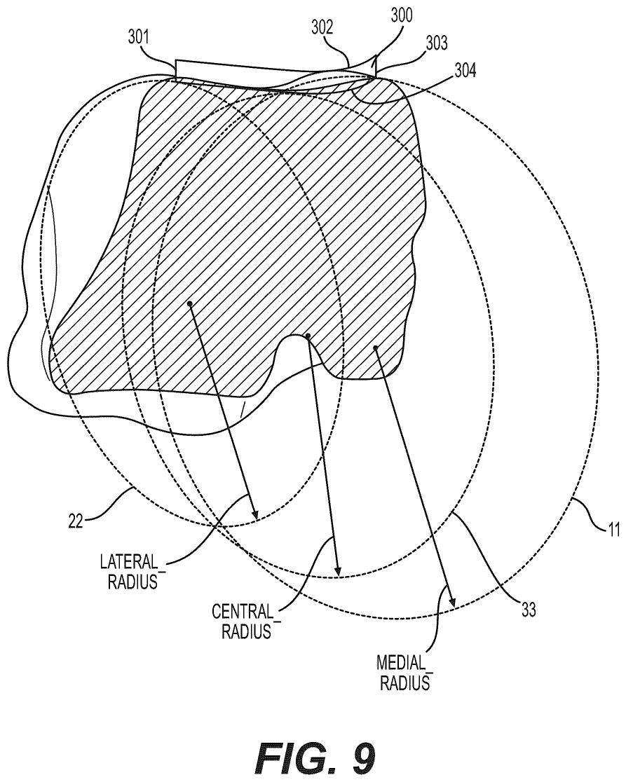

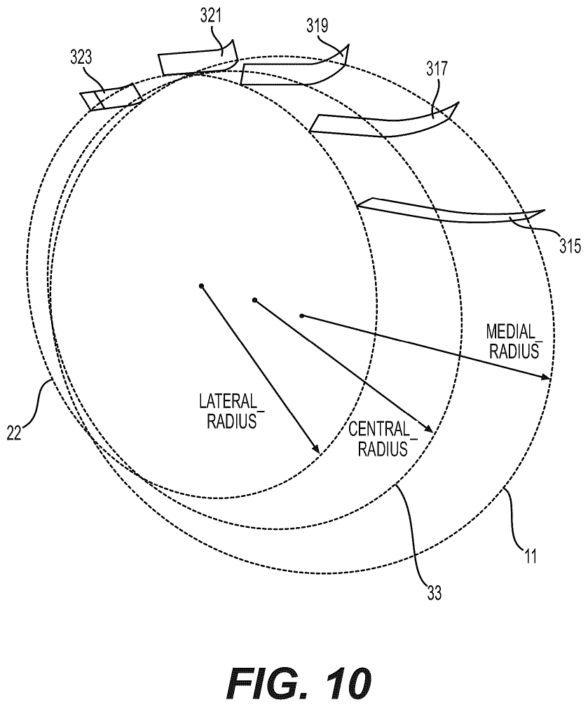

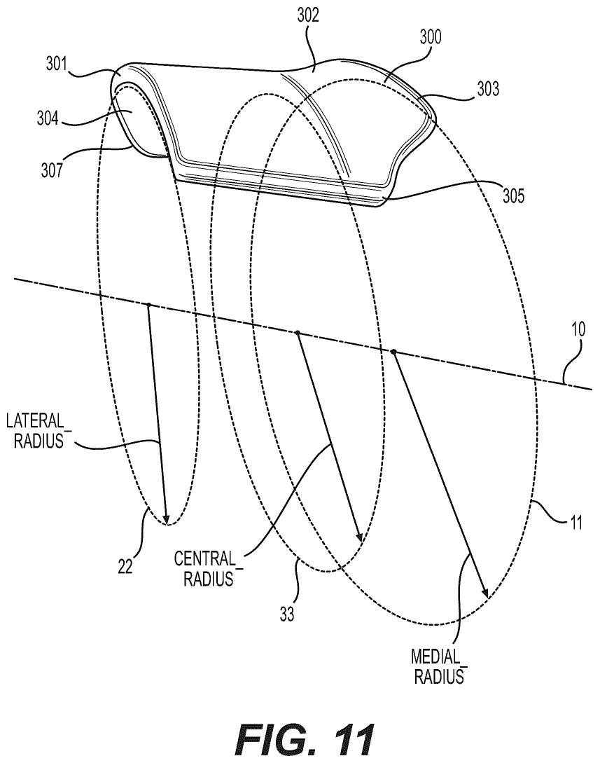

The talar component 300 of the prosthetic ankle of the present invention has a top surface 302 that preferably resembles certain contours of the top surface of the talus. Referring to FIG. 9, the talar component 300 of the present invention may be designed by generating frontal plane cross-sections of the talar component 300 in one or more frontal planes following the curvature of the top surface of the truncated cone 5 obtained as described above using the medial circle 11, lateral circle 22 and central circle 33. Five different frontal plane cross-sections 315, 317, 319, 321 and 323 of the talar component 300 are shown in FIG. 10. Cross-section 315 is the anterior-most cross-section and cross-section 323 is the posterior-most cross-section. These cross-sections 315, 317, 319, 321 and 323 may be connected to form a talar component 300 as shown in FIG. 11.

In some embodiments, the top surface 302 of the talar component 300 may have a larger curvature on the medial side 303, as compared with the curvature on the lateral side 301, as viewed in a frontal plane cross-section. As shown in FIG. 9, the top surface 302 on the lateral side 302 of talar component 300 may be flat or almost flat. In some other embodiments, the curvature of the top surface 302 of talar component 300, as viewed in a frontal plane cross-section may be uniform or substantially uniform from the medial side 303 to the lateral side 301. In some cases, the frontal-plane curvature in the cross-section taken proximate to posterior end 307 may change from concave to convex relative to a location above top surface 302.

The curvature of the top surface 302 of talar component 300 as viewed in a frontal plane cross-section may be described by a creating a frontal plane circle with its center located above top surface 302 and which is best fit to the curvature of the top surface 302. The radius of such a best fit frontal plane circle may vary in different frontal plane cross-sections 315, 317, 319, 321 and 323 of the talar component 300. In some embodiments the radius of the frontal plane circle is smaller when measured in a frontal plane proximate to the anterior end 305 of the talar component 300 than the radius of the frontal plane circle when measured proximate to the posterior end 307 of the talar component 300. A larger radius of the frontal plane circle corresponds to less curvature in that frontal plane.

In some embodiments, the radius of such a best fit frontal plane circle taken proximate to the anterior end 305 of talar component 300 may be in the range of 24 mm to 180 mm, or 35 mm to 165 mm, or from 50 to 150 mm. The radius of such a best fit frontal plane circle taken at a central location between the anterior end 305 and the posterior end 307 of the talar component 300 may be in the range of 25 mm to 300 mm, or from 40 mm to 280 mm, or from 60 mm to 250 mm. The radius of such a best fit frontal plane circle taken proximate to the posterior end 307 of talar component 300 may be in the range from 25 mm to infinity, or from 40 mm to infinity, or from 60 mm to infinity. When the radius of the best fit frontal plane circle is infinite, this indicates that the top surface 302 is flat or changes from concave curvature to convex curvature, relative to a location above top surface 302, as viewed in that frontal plane cross-section.

Referring to FIG. 10, five frontal plane cross-sections of the talar component 300 are shown. From a comparison of the cross-section taken at the posterior end 307 of talar component 300 and the cross-section taken at the anterior end 305 of talar component 300 it can be seen that in the depicted embodiment of the invention the top surface 302 of talar component 300 has the most curvature at the anterior end 305 of talar component 300 and has less curvature at the posterior end 307 of talar component 300. At the posterior end 307, top surface 302 of talar component 300 may be flat or substantially flat, as viewed in a frontal plane cross-section. Relative to a location above top surface 302 of talar component 300, the curvature in these frontal plane cross-sections is concave.

This variation of the average radius of concave curvature of top surface 302 in the anterior posterior direction is used to more closely approximate the actual shape of the talus of a subject since a variation in average radius of curvature is also present in the human talus. As a result, this feature may provide a closer approximation of the actual motion of an ankle relative to a prosthetic ankle without this feature. This feature can help to provide stability and smooth motion in inversion and eversion.

As shown in FIG. 11, talar component 300 has a top surface 302 that resembles certain aspects of the modeled top surface of the talus. Of the three circles 11, 22, 33 that are used model the top surface 302 of the talar component 300, the radius of the medial circle 11 is larger than the radius of the lateral circle 22. The ratio of the radius of medial circle 11 to the radius of lateral circle 22 may be in the range of about 1.5:1 to 1.01:1, or from 1.35:1 to 1.1:1, or from 1.3:1 to 1.15:1. In one embodiment, the ratio of the radius of the medial circle 11 to the radius of the lateral circle 22 is about 1.25:1-1.2:1.

It is not necessary to take actual measurements of the human talus to develop the circles 11, 22 and 33. Rather, these circles can be developed from the information provided herein rather than by actual measurement. In practice, it may be advantageous to provide different sizes of implants that can be selected for particular patients or, the technique of the present invention can also be used to make customized implants tailored to specific patients by taking actual measurements of the patient's ankle.

Talar component 300 of one embodiment of the invention has a saddle-shaped structure that has curvature on both its top surface 302 and bottom surface 304. Top surface 302 has convex curvature relative to a location above the top surface, in the direction from anterior end 305 to posterior end 307 as viewed in a sagittal plane cross-section and concave curvature relative to a location above the top surface, in the direction from lateral side 301 to medial side 303, as viewed in a frontal plane cross-section, which, in combination form the saddle shape of top surface 302 of talar component 300.

The convex curvature of top surface 302 in the medial plane 1 has a larger average radius of curvature than the average radius of curvature of the convex curvature of top surface 302 in the lateral plane 2 of the talar component 300 as indicated by the fact that the radius of the lateral circle 22 is smaller than the radius of the medial circle 11. The top surface 302 of talar component 300 thus resembles a truncated conical surface oriented so that the cone has its apex on the lateral side 301 of the ankle. The top surface 302 of the talar component 300 thus approximates the native truncated conical surface shape of the trochlear surface of the talus.

The average radius of curvature of the top surface 302 in a sagittal plane cross-section is obtained by averaging the radius of curvature over a major portion of the top surface 302 of the talar component 300 in a sagittal plane, which major portion constitutes from at least greater than half of the length of the top surface 302 to the entire length of the top surface 302 of the talar component 300 in the anterior/posterior direction, or alternatively at least 80% of the length of the top surface 302 or at least 90% of the length of the top surface 302 of the talar component 300 in the anterior/posterior direction.

The particular curvature of the top surface 302 of talar component 300 of the present invention provides significant benefits relative to existing prior art devices. For example, the provision of an average radius of concave curvature of the top surface 302 on the medial side 303 of the talar component 300 that is larger than the average radius of concave curvature on the lateral side 301 of the talar component 300, as viewed in a sagittal plane cross-section, provides a shape of a truncated conical surface with the apex of the cone oriented in a substantially lateral direction. As a result, the device of the present invention allows motion that closely resembles supination and allows an approximation of the movement of an actual ankle, particularly in the lateral and medial directions as well as in plantar flexion.

A result of these features of the present invention is the provision of a prosthetic ankle wherein the truncated conical shape 5 used to approximate the talus can be extended to provide a cone 7 with the apex 6 oriented substantially in a lateral direction. By “substantially in a lateral direction” is meant that the apex 6 of the cone 7 may be oriented at an angle from the lateral direction. As a result, the talar component 300 of the present invention can be fabricated to ensure that the device is oriented similarly to the actual talus of a particular subject or based on information obtained from several subjects.

In certain embodiments, axis 10 of cone 7 is skewed upward and/or in the anterior direction, relative to the lateral direction. The angle between axis 10 and the lateral direction, as viewed in three dimensions, is referred to as the total conic offset angle, which may be in the range of 0° to or 3° to 40°, or 7° to 38°.

The angle between the axis 10 and the lateral direction when projected in an horizontal plane, is referred to as the horizontal conic offset angle, which may be in the range of from 0° to 35°, or from 3° to 30°, or from 5° to 28°.

The angle between axis 10 and the lateral direction when projected in a vertical plane, is referred to as the vertical conic offset angle, which may be in the range of from 0° to 40°, or from 3° to 37°, or from 5° to 35°.

In certain embodiments, the top surface 302 of the talar component 300 may resemble a truncated cone 7 having an axis 10 along line A-A of FIG. 17, and being further defined by line B-B extending perpendicular to the medial circle 11 of the cone and through the center of the medial circle 11 (FIG. 17). In this embodiment, cone 7 represents the top surface of the talar component 300 and has a total axis offset angle in a range from 0° to 40°, or from 5° to 35°, or from 10° to 30°, or from 16° to 24°. The coronal axis offset angle is in a range of from 0° to 38°, or from 6° to 32°, or from 10° to 28°, or from 15° to 23°. The transverse axis offset angle is in a range of from 0° to 20°, or from 4° to 16°, or from 6° to 14°.

The bottom surface 304 of talar component 300 preferably has a generally concave curvature in the anterior to posterior direction, as viewed in a sagittal plane cross-section from a location below bottom surface 304. The concave curvature is designed to be suitable for implantation onto the talar dome. However, a skilled person will appreciate that the bottom surface 304 of talar component 300 may have a variety of different shapes so long as the shape of the bottom surface 304 of talar component 300 is adapted to approximately or exactly fit with the surface of the bone of the talar dome in accordance with principles of the present invention.

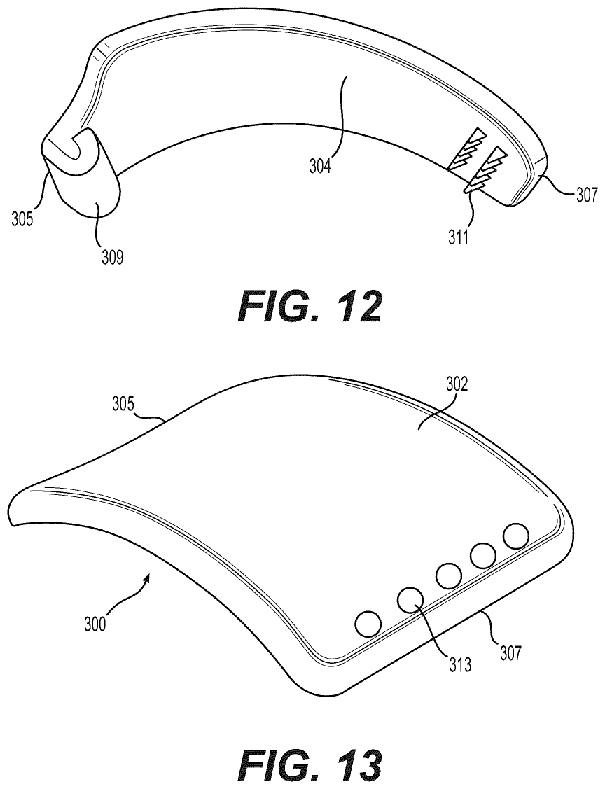

In one embodiment, bottom surface 304 of talar component 300 has at least one protrusion, ridge or peg 309 that extends downwardly from bottom surface 304. Such protrusions, ridges or pegs 309 are designed to fit with the shaped surface of the talar dome and provide an additional structure that can be used to secure talar component 300 to talus. The position of the protrusion(s), ridge(s) or peg(s) 309 of the bottom surface 304 may vary. In one embodiment, a protrusion, ridge or peg 309 may be located proximate to the anterior end 305 of the talar component 300 as shown in FIG. 12. In another embodiment, one protrusion, ridge or peg 309 is located at the anterior end 305 of bottom surface 304, and spikes 311 are located on posterior end 307 of the bottom surface 304 as shown in FIG. 12. The spikes 311 are for penetrating into the talus thus affixing the talar component 300 to the talus. In yet another embodiment, holes 313 may be provide proximate to posterior end 307 of the talar component 300 as shown in FIG. 13. Holes 313 may be used to fix the talar component 300 to the talus using, for example, screws or other suitable attachment devices. In this embodiment, a ridge, protrusion or peg 309 may also optionally be located on the anterior end 305.

Alternatively or additionally, the bottom surface 304 may be affixed to talar component using joining means other than the protrusions, ridges or pegs 309. Conventional joining means may include means such as adhesives, screws, friction fit, form fit and/or any combination thereof. Such means may include bone cement such as poly(methyl methacrylate), nails, plugs and any other suitable means known to skilled persons for affixing talar component 300 onto the talus.

Referring to FIG. 8A, the tibial component 100 of the prosthetic ankle may have a bottom surface 104 configured with a shape and curvature that substantially fits with and complements aspects of the curvature of top surface 302 of the talar component 300. For example, the anterior end of the tibial component 100 may be aligned with and complement anterior end 305 of the talar component 300 with substantially matching curvatures, while the posterior end of the tibial component 100 may be aligned with and complement posterior end 307 of talar component 300 with substantially matching curvatures. With this configuration, tibial component 100 can frictionally engage and move along top surface 302 of talar component 300. This configuration allows internal and external rotational motion of the ankle joint with the prosthetic ankle, as well as dorsiflexion and plantar flexion. The width of prosthetic ankle, from the medial side to the lateral side, may be in the range of from 15 mm to 35 mm, or from 18 mm to 33 mm, or from 20 mm to 30 mm.

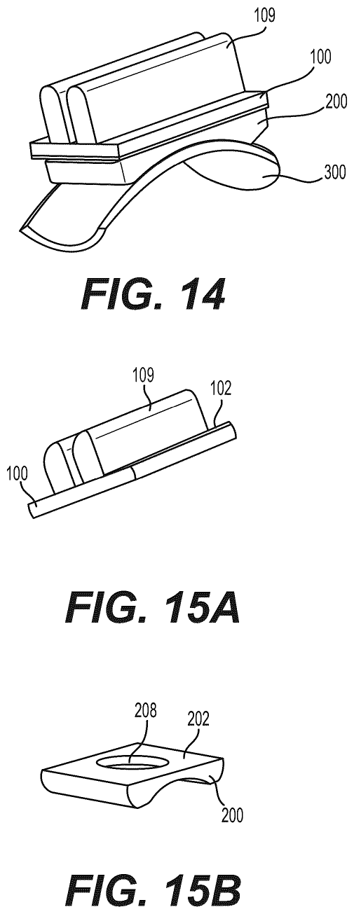

Top surface 102 of tibial component 100 is adapted for affixation to the tibia. Thus top surface 102 may have a shape or configuration that approximately or exactly fits with the lower surface of the prepared/carved tibia. In one embodiment, as shown in FIG. 8A, top surface 102 may have one or more spikes 108 adapted for fixing the tibial component 100 onto the tibia. In another embodiment, referring to FIG. 14, the tibial component 100 may have one or more protrusions 109 extending in an anterior/posterior direction that are configured to fit within the similarly shaped recesses that have been made in the prepared surface of the tibia. The spikes 108 and protrusions 109 serve to stabilize motion of tibial component 100 relative to the prepared distal tibial surface and provide greater surface area for bony ingrowth or cement fixation of tibial component 100 to the tibia. These protrusions or ridges 109 may be tapered, from more narrow on a medial side to wider on a lateral side, so as to create a more secure and stable fit.

Alternatively or additionally, top surface 102 of tibial component 100 may be affixed to the tibia using means other than protrusions 109 or spikes 108. Such means may include bone cement such as poly(methyl methacrylate), nails, plugs and any other means known to a skilled person to be useful for affixing the tibial component 100 onto the tibia. The tibial component 100 of the present invention is designed to be joined to the tibia during the implantation procedure using conventional joining means such as adhesives, screws, friction fit, form fit and/or any combination thereof.

An example of talar component 300 is shown in FIG. 8B where top surface 302 of talar component 300 can be seen. Bottom surface 304 of talar component 300 and/or top surface 102 of tibial component 100 may be coated with a substance to enhance bony ingrowth or cement fixation.

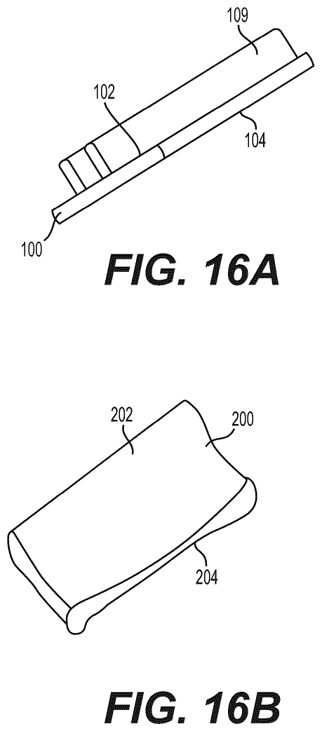

In some alternative embodiments, the prosthetic ankle of the present invention includes a third component, namely, a bearing component 200 as shown in FIG. 14. In these alternative embodiments, the talar component 300 is the same as described above. The top surface 102 of the tibial component 100 is also the same as described above. However, the bottom surface 104 of the tibial component 100 may be flat as shown in FIG. 16A or have another suitable configuration for frictional engagement with top surface 202 of bearing component 200.

Bearing component 200 is designed for location between tibial component 100 and talar component 300 to provide bearing surfaces that allow relative motion between tibial component 100 and talar component 300. Top surface 202 of bearing component 200 may also be flat as shown in FIG. 16B, to match a flat bottom surface 104 of tibial component 100. Bottom surface 204 of bearing component 200 may be adapted to substantially match and/or complement the shape of top surface 302 of talar component 300. This configuration allows bearing component 200 to cooperatively engage both tibial component 100 and talar component 300 by frictional engagement. This enables relative movement between bearing component 200 and both tibial component 100 and talar component 300.

The thickness of bearing component 200 may be varied for adaptation of the prosthetic ankle for subjects having differences in the anatomy of their ankles. A suitable thickness of the bearing component 200 may be determined by examination of the ankle of the subject for which the prosthetic ankle is intended.

Selection of the thickness of bearing component 200 permits adjustment of the overall height of the prosthetic ankle. Thus, the present invention may provide a prosthetic ankle that is adaptable, depending on the thickness of the bearing component 200. This provides options for dealing with different clinical situations. Ultimately, the goal will be to use a prosthetic ankle that balances considerations of providing maximum range of movement, minimizing wear and enhancing the longevity of the implant.

In some embodiments, bearing component 200 may be semi-constrained. This may be achieved by using a tibial component 100 having a bottom surface 104 with one of a variety of forms of curvature that are designed to provide varying degrees of constraint on the motion relative to the underlying bearing component 200. A skilled person will appreciate that the curvature of bottom surface 104 of tibial component 100 and the curvature of top surface 202 of bearing component 200 may be altered in these embodiments to achieve the desired degree of constraint of motion.

To illustrate this, bottom surface 104 of tibial component 100 can be curved so that bottom surface 104 is configured for fitting with a curved portion of top surface 202 of bearing component 200. In one embodiment, shown in FIGS. 15A-15B, a plug 106 may be formed on bottom surface 102 of tibial component 100. Plug 106 is adapted to engage a corresponding recess 206 on top surface 202 of bearing component 200. Such a plug 106 can be located at any suitable location but in one embodiment is centrally located in bottom surface 104 of tibial component 100. The plug 106 can be of any suitable size, shape or configuration as desired and as can be appreciated by those of skill in the art in order to allow for a desired range of motion as the tibial component 100 and the bearing component 200 interact and articulate with one another.

In some other embodiments, top surface 202 of bearing component 200 may be bonded or mechanically attached to bottom surface 104 of tibial component 100. This may also provide a desired level of constraint on relative motion between the bearing component 200 and tibial component 100. More means of constraining or semi-constraining the mobility of bearing component 200 relative to tibial component 100 are disclosed in WO 2006/023824, which is hereby incorporated by reference in its entirety.

In these semi-constrained bearing embodiments, top surface 302 of talar component 300, bottom surface 204 of bearing component 200, and top surface 102 of tibial component 100 may be the same as in the unconstrained embodiments described above.

Tibial component 100 and talar component 300 may be made of the same or different materials and the materials may be selected from any appropriate material suitable for the surgical environment. High density, ultra-high molecular weight polyethylene is a suitable material for fabrication of these components. This material is widely used in other surgical devices and characterized by excellent wear resistance and a low coefficient of friction. Metallic alloys that are biocompatible are also suitable materials for the tibial and talar components 100, 300 of the present invention. Exemplary materials include titanium alloys and cobalt chrome alloys. Stainless steel or ceramics may also be used to fabricate the two components.

Bearing component 200 of the present invention is preferably made of a synthetic plastic material such as a high density, ultra-high molecular weight polyethylene that provides a low coefficient of friction and excellent wear resistance. The high density, ultra-high molecular weight polyethylene used in the present invention may have an extremely long chain with a molecular weight generally between 1 and 10 million Daltons, or between 2 and 6 million Daltons.

It will be understood by a skilled person that tibial component 100, bearing component 200, and talar component 300 will be made in left and right mirror-image embodiments and may be made in different sizes to accommodate subjects of different sizes. The size of the device does not constitute a limitation of the present invention. It is believed, for example, that a wide range of subjects can be accommodated by providing each of these components in three sizes. Bearing component 100 can also be made in several different thicknesses for the reasons given above.

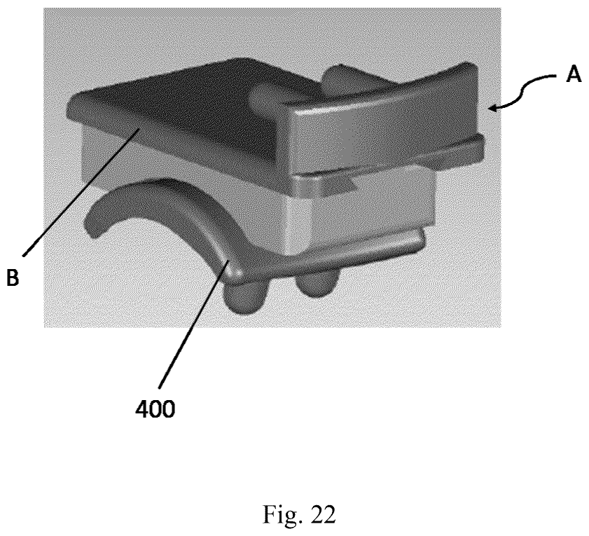

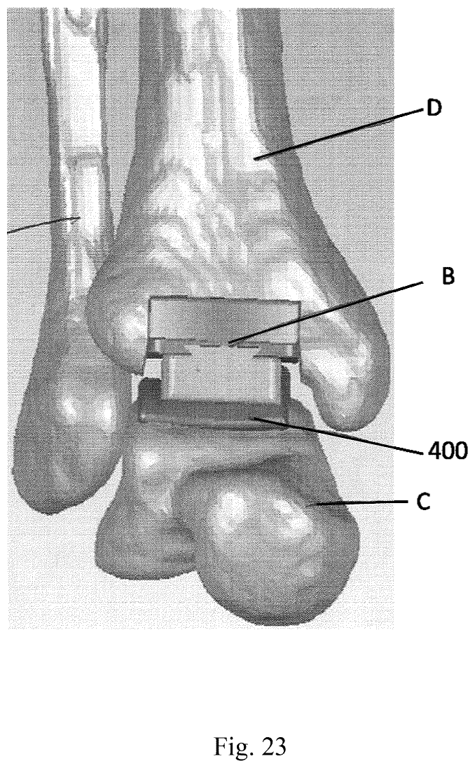

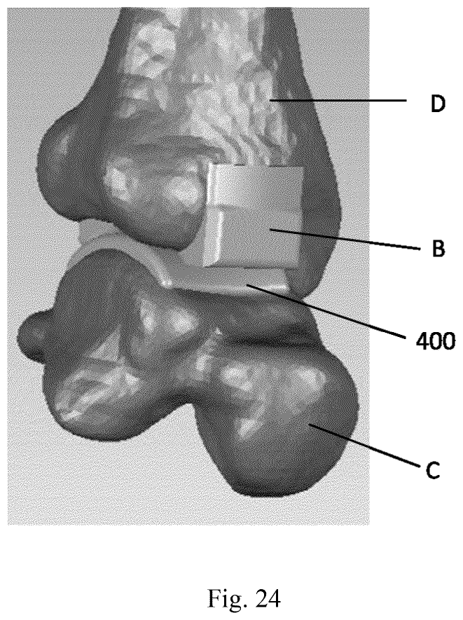

FIG. 22, shows a talar component 400 that is an anatomically fitted component of a total ankle replacement system A comprising the talar component 400 and a tibial component B and optionally a bearing component as described above. As discussed above, these components may be made of any material that is known in the art with preferable materials listed previously. FIG. 23 shows the anatomically fitted talar component 400 covering the surface of talar dome C. The tibial surface of the talar component interacts with a part B of the ankle replacement system affixed to the tibia D. FIG. 24 shows a side view of the anatomically fitted talar component 400 covering the surface of the talar dome C. In the view of FIG. 24, the fibula has been removed so that the mating surface between the talar dome C and the talar component is more easily seen, and the the talar dome with minimal or no bone resection is visible.

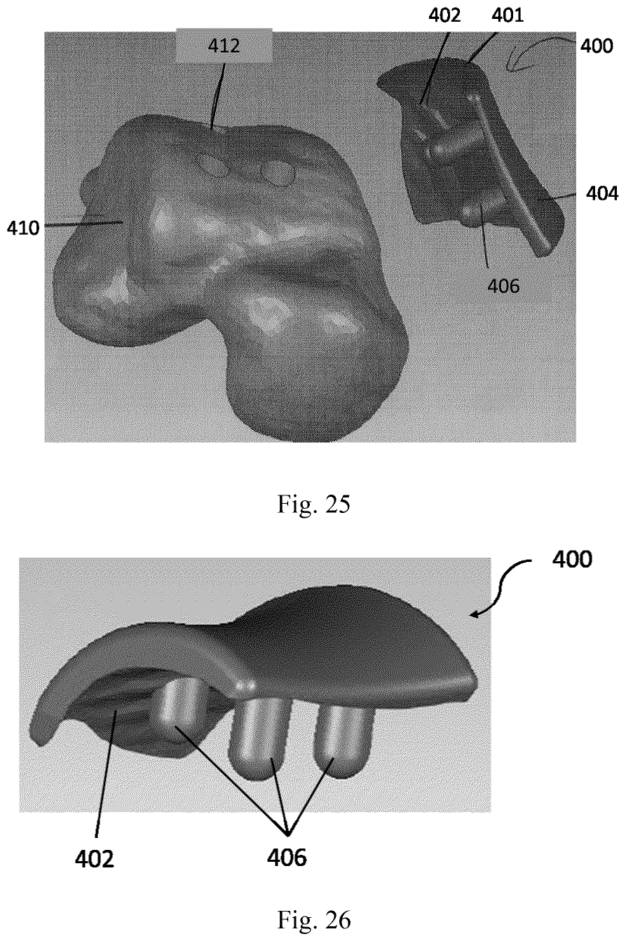

The anatomically fitted talar component 400 includes a body 401 having a talar surface 402, as shown in FIGS. 25,27B, and 28. The talar surface 402 has a portion contoured to approximately or exactly fit with a surface portion of a three-dimensional rendering of the talar dome 410. The portion of the talar surface 402 includes at least 50% talar surface, defined as the surface of the talar component that faces the talus. Preferably, the portion of the talar surface 402 includes at least 75% of the talar surface, more preferably, the portion of the talar surface 402 includes at least 90% of the talar surface, and most preferably, the portion of the talar surface 402 includes the entire talar surface.

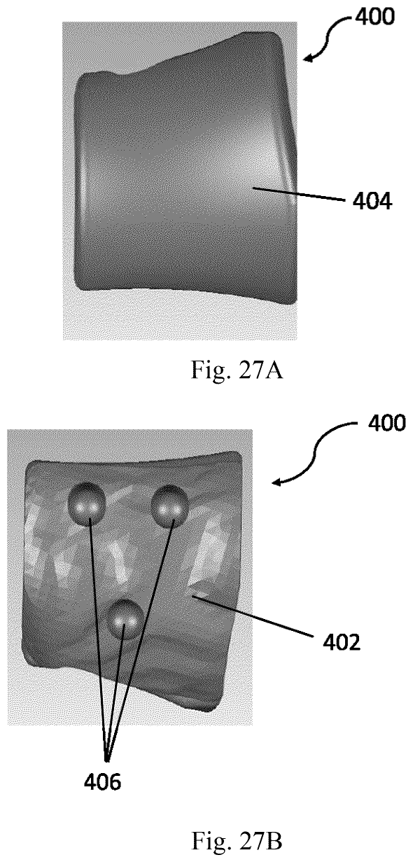

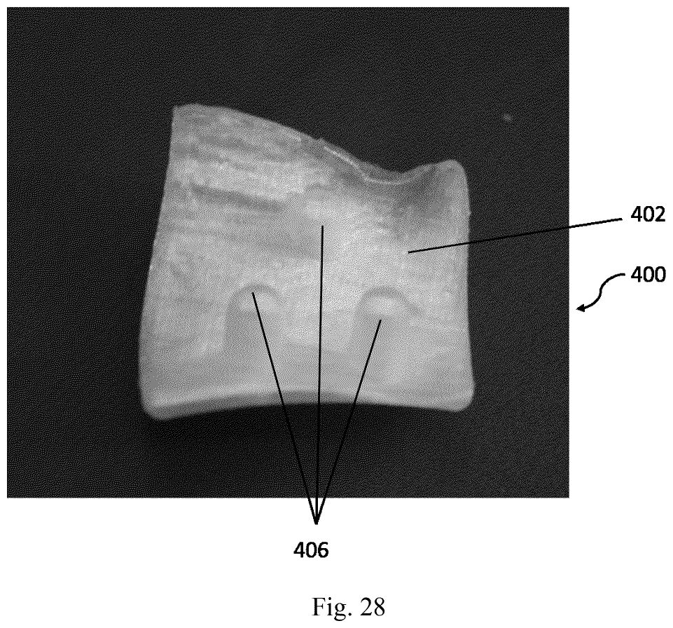

The talar component 400 also has a tibial surface 404 as shown in FIG. 27A configured for forming a joint with a second component of an ankle replacement system, which in a preferred embodiment is a tibial component, but may also be an intermediate component between the talar and tibial components.

As used herein, the term, “approximately” as used, for example, in the phrase, “approximately fit”, means not exact. Thus, a surface portion that approximately fits another surface portion does not exactly fit. As a result, the distance between surface portions that approximately fit one another may vary by up to 0.5 mm, or up to 0.25 mm, or up to 0.1 mm.

To create the talar surface of the talar component image data is obtained from a patient's ankle. Any known technology for obtaining suitable image data can be used for this process, including one or more of the following data gathering systems, MM, CT scans, X-rays, etc. A three-dimensional rendering of the bone of the talar dome is created from the image data. The three dimensional model may be digital, or can be formed into a physical model using model forming technologies from a digital model, such as 3D printing, or cast molding. Preferably, the model is physically formed using 3D printing techniques, which allows for the details of the bone to be displayed precisely as they occur on the patient's bone, and which may also be customized to include any changes to the bone that may be necessary due to disease or injury of the joint.

In an alternative preferred embodiment, a 3D model of the talar component is generated from the three-dimensional rendering of the talus and then the talar component is fabricated, for example, by 3D printing techniques.

Although the three-dimensional rendering is preferably as exact a replica, or as close an approximation of the talus of the patient that the state of the technology in the art for imaging will allow, there are certain instances where modification of the image-based data or the three-dimensional rendering of the bone of the patient's talus may be desirable prior to the creation of the talar component. Such instances include, but are not limited to, correcting the data to compensate for either a chronic or acute injury that occurred to the patient's ankle at some time in the past. Alterations of the model may also be employed to correct alignment issues that may be present in the patient's ankle due to bone deformities, or bone degradation from disease. The model may be altered to provide a desired shape of the patient's talus or by alteration of the model of the patient's talus after it has been created from the originally obtained image data of the patient's ankle.

Alteration of the image data or the three-dimensional rendering can be used to compensate for injury, disease or damage to the patient's talus. Such compensation may involve one or more changes in the fabricated talar component 400 that will result from alteration of the model of the patient's talus prior to fabrication of the talar component 400. Such alterations to the model of the patient's talus may include changes in the talar surface to account for altered surface sections of the model of the patient's talus prior to inserting the implant, as well as providing corrective measures to ensure proper alignment replacement joint elements that may provide improved ankle motion and use.

This is an important feature of the invention since it allows the talar component 400 to be specially fabricated for a desired future configuration of the patient's ankle to be used in the ankle replacement. As a result, the present invention facilitates implementation of desirable corrective measures as part of the ankle replacement procedure to thereby potentially improve the patient's mobility and ensure a successful and reliable implantation of a total ankle replacement system while minimizing or avoiding resection of the subchondral bone of the patient's talus.

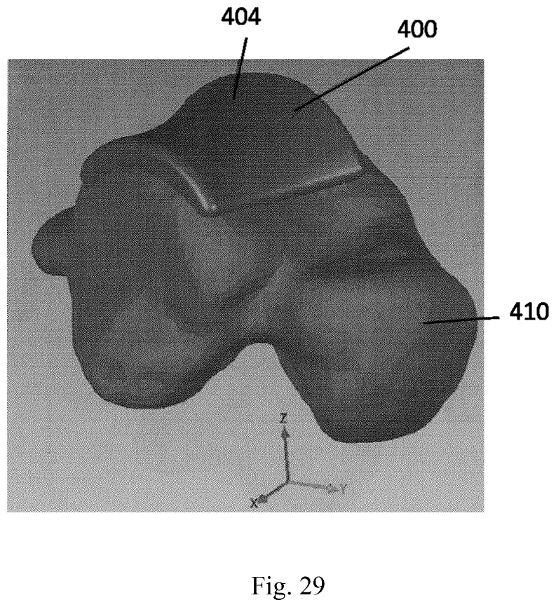

The talar surface 402 of the talar component 400 is formed based on the geometry of the three dimensional rendering 410. The talar surface 402 of the talar component 400, which corresponds to the inferior side of the talar component, interfaces with the trochlea of the talus as show in FIG. 29, and approximately, or exactly fits the geometry of the bone, as shown in the three-dimensional rendering. In a preferred embodiment, none of the subchondral bone of the talus is removed prior to affixing the talar component to the bone. In another embodiment, only portions of the cortical bone of the talus are removed leaving the cancellous bone. In such case, no more than 75% of the surface of the talar dome is removed.

In another preferred embodiment, to better secure the talar component 400 to the talus, one or more protrusions 406, or pegs, are formed on the talar surface 402. These protrusions 406 are shaped to fit a corresponding resected portion 508 of the bone of the talar dome C. In this embodiment, a portion of the subchondral bone of the talus of the patient is resected to provide recesses to receive the protrusions or pegs 406. The resected portion 412, or recess(es), may be added to the digital model 410 through the use of digital alteration prior to formation of the three dimensional model, or can be manually drilled into the physical model after it is formed. Based on the modeled resected surface portion of the bone of the talus, the corresponding protrusions 406 on the talar surface 402 may be formed. A portion of the talar surface 402 of the talar component of this embodiment approximately or exactly fits with the resected surface portion of the corresponding surface portion of bone of the talar dome C. Preferably, there are three protrusions as shown in FIGS. 25, 26 and 27B.

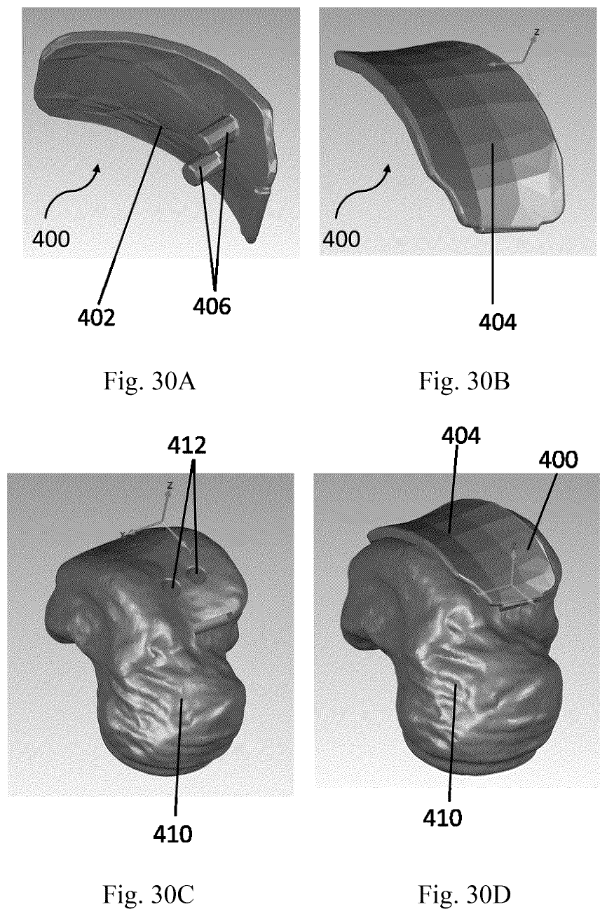

Also preferred is a talar component 400 having two protrusions 406 as shown in FIGS. 30A-D. In this embodiment of the talar component 400, there are only two protrusions 406 located on the front half of the talar surface 402. These two protrusions 406 are secured in two holes 412 that are resected into the talar dome C. The process of altering the three-dimensional model, and the talar dome are the same as described above.

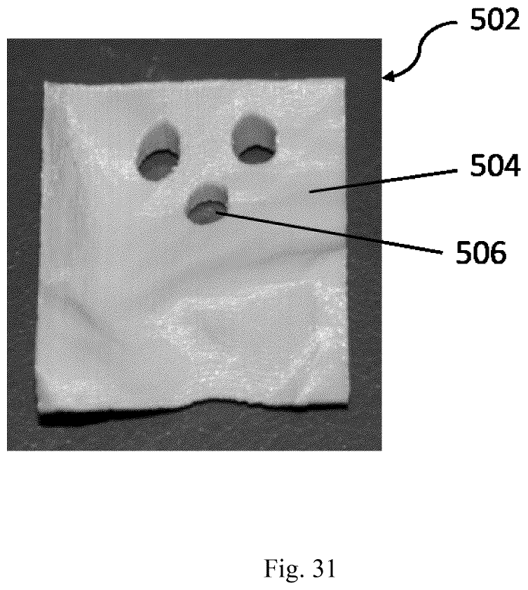

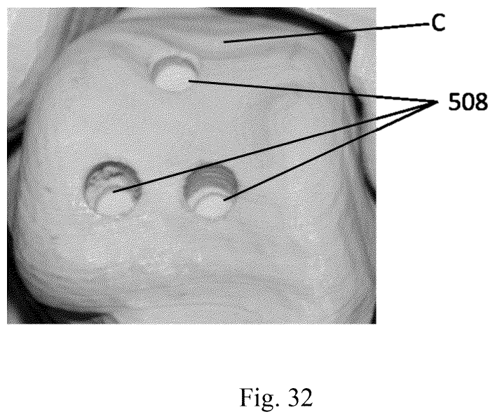

A template 502, shown in FIG. 31 for drilling the necessary recesses 508 into the patient's talar dome C, as shown in FIG. 32 may also be created from the three-dimensional model 410.

Other affixing methods are also contemplated for use in combination or by themselves. For example, pins, screws, bone cement, and hydroxyapatite, or other compounds to promote osseo-integration can be implemented alone or in combination with each other to secure the talar component to the subchondral bone of the patient's talus. If these methods of securing require additional features protruding from the talar surface, or recessed into the talar surface, such as the pegs described above, they can be included in the three dimensional model, so that they are represented in the formed talar component. Further, any desirable templates can be created to facilitate removing minimal amounts of bone from the patient's talus for this purpose.

The superior side of the talar component includes a tibial surface, as opposed to the inferior surface of the talar component that approximately or exactly fits with the bone surface of the talar dome with the exception of any holes that were drilled into the bone to secure the talar component, articulates with the tibial component of a TAR. The complete TAR with its talar and tibial components can have any suitable geometry for the articulating surfaces located between the components. However, a preferred tibial surface has the geometry as described above for forming a joint with the tibial component.

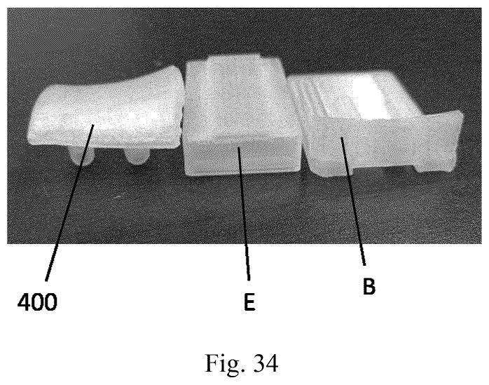

FIG. 34 depicts a total ankle replacement system having a talar component 400 according to one embodiment of the present invention. The talar component 400 was formed from image data using 3D printing technology. In this preferred embodiment the talar component interacts with a bearing component E, which is connected to the tibial component B.

A method of forming the talar component 400 of a total ankle replacement system A is also part of the present invention. The method involves obtaining image data of the talar dome of a patient. The image data may be obtained through any known image gathering system, such as computer tomography (CT), magnetic resonance imaging (MRI), X-ray, etc. The image data that is obtained is then used to create a three dimensional model 410 of the talar dome. A talar component body 401 is then formed from the three dimensional model 410. The talar component body 401 has a talar surface that approximately or exactly fits with a portion of the surface of the talar dome of the three-dimensional model.

The talar surface of the three dimensional model may be modified to compensate for injury or disease of the talus. The modification of the surface of the three dimensional model may either be made through altering the image data that was obtained prior to the formation of the model or altering the surface of the model after formation of the model, but prior to forming the body of the talar component. Such alterations can be done on a digital model created from the image data or a physical model created from the image data.

Also prior to forming the talar component, one or recesses 412 in a surface of the three dimensional model 410 may be created if it is desirable to provide such recesses 412 to help secure the talar component. When the talar component is formed from the three dimensional model, the recesses in the model are used to form one or more protrusion(s) 506 on the talar surface 502 of the talar component 400 that is aligned with the recess(es) 412 of the three dimensional model 410.

Also, a template as shown in FIG. 31 may be formed from the three-dimensional model having a surface 504 approximately or exactly fitting with the surface of the bone and containing at least one hole 506 at the location of each of the one or more recess(es) in the model. This template can then be used during the joint replacement procedure to resect portions of the talus C of the patient for the purpose of creating at least one recess 508 in the bone corresponding to the at least one protrusion 406 on the talar component 400.

The present invention also includes a method of attaching the talar component as described above to a talus of a patient. The method includes shaving one or more of the following items from the talus of the patient: the articular cartilage, osteophytes, and non-conforming portions of the talar dome to expose the bone of the talar dome. Preferably, the non-conforming portions of the talar dome represent less than 75% of the surface area of the talar component. It is desirable to remove, at most, a minimal amount of the subchondral bone of the talus, including the non-conforming portions, to provide the strongest connection between the remaining bone of the talus and the talar component. Preferably, none of the subchondral bone of the talus is resected. In another preferred embodiment, resection of the subchondral bone is done only to the extent required to form the one or more recess(es) 506 within the bone of the talus. By utilizing the image data of the specific patient's ankle, the resection of the bone can be minimized by conforming the mating surface of the patient specific talar component that is formed from the model.

In a preferred embodiment, one or more recesses 508 are created in the bone of the talar dome C. These recesses correspond to one or more protrusions 406 located on the surface 402 of the talar component 400. These recesses 406 are added to the surface 402 of the talar component 400 to enhance the attachment of the talar component 400 to the bone surface.

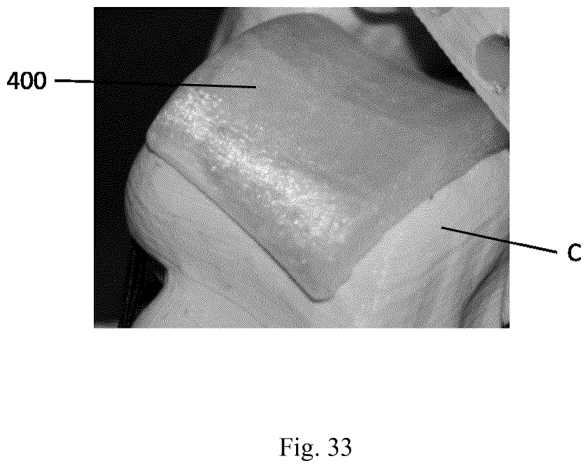

The talar component 400 is then secured to the prepared talar dome C as shown in FIG. 33 with the protrusions, if any, located within the recesses, if any. Although the use of protrusions and recesses in the bone are described, the talar component may be secured to the talar dome using any means known in the art, such as the use of pins, screws, bone cement, and hydroxyapatite, or other compound to promote osseo-integration can all be utilized alone or in combination with each other.

Although minimal resection of the bone is desired, in some cases it is necessary to remove additional parts of the bone due to injury or disease. In such cases, the talar component is designed to approximately or exactly fit a future prediction of the surface of the bone that includes the necessary alterations. Therefore, if the bone must be resected corresponding changes are made to the three-dimensional model of the talus to account for the resected bone and ensure that the talar component fits properly to the bone after resection. In such cases, a portion of the talar dome will be resected prior to securing the talar surface of the talar component to the talar dome.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meanings of the terms in which the appended claims are expressed.

1-9. (canceled)

10. A method of attaching a talar component to a talus comprising steps of:
shaving one or more of articular cartilage, osteophytes, and non-conforming portions of a talar dome to expose bone of the talar dome, wherein the non-conforming portions of the talar dome represent less than 75% of a surface area of the talar component;
creating one or more recesses in subchondral bone of the talar dome to correspond to one or more protrusions located on a talar surface of the talar component; and
securing the talar surface of the talar component to the talar dome with the protrusions located in the recesses.

11. The method of claim 10, wherein no resection of the talus is carried out other than creating the recesses.

12. The method of claim 10, wherein a portion of the bone of the talar dome is resected to compensate for injury to or disease of the talus prior to securing the talar surface of the talar component to the talar dome.

13. A method of forming a talar component of an ankle replacement system comprising steps of:
obtaining image data of talar dome;
using the obtained image data to create a three-dimensional model of the talar dome; and
forming a body having a talar surface that approximately or exactly fits with a portion of the surface of the three-dimensional model.

14. The method of claim 13, further comprising a step of:
modifying a portion of the talar surface of the three-dimensional model of the talar dome to compensate for injury to or disease of the talus prior to forming the body.

15. The method of claim 13, further comprising a step of:
altering the image data to compensate for injury or disease on the surface of the talar dome prior to using the image data to create the three-dimensional model of the talar dome.

16. The method of claim 13, further comprising a step of:
creating at least one recess in a surface of the three-dimensional model prior to forming the body.

17. The method of claim 16, further comprising a step of:
altering the image data to include the at least one recess prior to using the image data to create the three-dimensional model of the talar dome.

18. The method of claim 13, further comprising a step of:
forming at least one protrusion on a portion of the talar surface of the body.

19. The method of claim 18, wherein each said at least one protrusion aligns with at least said recess in the surface of the three dimensional model.

20. The method of claim 13 wherein the talar component for use in an ankle replacement system comprises:
a body including:
a talar surface having a portion contoured to approximately or exactly fit with a surface portion of a three-dimensional rendering of bone of a talar dome; and
a tibial surface configured for forming a joint with a second component of the ankle replacement system.

21. The method of claim 13, wherein the three dimensional rendering includes cortical bone of the talar dome.

22. The method of claim 13, wherein the three dimensional rendering includes a portion of cancellous bone of the talar dome that is exposed by resection of a portion of cortical bone of the talar dome.

23. The method of claim 20, wherein the portion of the talar surface approximately or exactly fits with a resected surface portion of the corresponding surface portion of the bone of the talar dome.

24. The method of claim 21, wherein the talar surface further comprises at least one protrusion shaped to fit a resected portion of bone of the talar dome.

25. The method of claim 20, wherein the portion of the talar surface exactly fits with the corresponding surface portion of the three dimensional rendering of the bone of the talar dome.

26. The method of claim 20, wherein the portion of the talar surface approximately fits with the corresponding surface portion of the three dimensional rendering of the bone of the talar dome or approximately fits with the corresponding surface portion of the bone of the talar dome.
